(19) United States
(12) Patent Application Publication
(10) Pub. No.: US 2017/0296066 A1
(43) Pub. Date: Oct. 19, 2017

(54) METHOD FOR QUANTIFYING WOUND INFECTION USING LONG-WAVE INFRARED THERMOGRAPHY

(71) Applicant: Jane E. Spahn, Carmel, IN (US)

(72) Inventors: James G. Spahn, Carmel, IN (US); Thomas J. Spahn, (US); Kadambari Nuguru, Indianapolis, IN (US)

(21) Appl. No.: 15/487,477

(22) Filed: Apr. 14, 2017

Related U.S. Application Data
(60) Provisional application No. 62/322,490, filed on Apr. 14, 2016.

(51) Int. Cl. A61B 5/01; A61B 5/00; G06T 7/00

(52) U.S. Cl. CPC A61B 5/015; A61B 5/7282; A61B 5/0077; G06T 7/0012; A61B 5/0075; A61B 5/445; A61B 2576/00; G06T 2207/30096

(57) ABSTRACT

A method of using long wave infrared thermography (LWIT) to quantify the characteristic temperature changes associated with infected wounds to accurately confirm the existence of or absence of infection.

### PRIORITY CLAIM AND CROSS-REFERENCE TO RELATED APPLICATION(S)

This application claims the benefit under Title 35, U.S.C. §119(e) of U.S. Provisional Patent Application Ser. No. 62/322,490 filed Apr. 14, 2016, entitled METHOD FOR QUANTIFYING WOUND INFECTION USING LONG-WAVE INFRARED THERMOGRAPHY.

### BACKGROUND

### 1. Field of the Invention

The present invention relates to thermography and, more particularly, to the use of quantitative analysis of long wave infrared thermography (LWIT) may be a useful tool to objectively and reliably identify a wound infection.

### 2. Description of the Related Art

The challenge of correctly identifying and appropriately treating wound infections has confounded clinicians since the earliest days of medicine. One of history's oldest surviving documents, the famous Edwin Smith papyrus, addressed some of the issues associated with wound management. Despite the significant increases in medical knowledge over the past several thousand years, wound management continues to challenge the medical community. This includes such basic issues as how to classify wound infections. Recently, an article in the Journal of the American College of Surgeons, found that concordance between the electronic medical record and the operative note for surgical wound classification at various institutions was only 56%. Such data indicate variability in the ability to objectively define, detect and document wound infections validating the historical difficulty that medical practitioners have had throughout the centuries in determining the presence of a wound infection.

The inability to reliably make an accurate diagnosis of wound infection complicates treatment, and increases the cost of care, with surgical site infections (SSI) costing about $21,000 per occurrence. SSI is one of the five most common hospital acquired infections and is estimated to cost the U.S. health care system almost $10 billion a year. The high cost of wound infections underlies the prevalence of wound infections and its elevated morbidity rate. According to the Centers for Disease Control, the morbidity rate for inpatient surgical site infection (SSI) reported from 2006-2008 was 3%. In 2010, a multistate point-prevalence survey of health care-associated infections, SSI was cited as the cause for 21.8% of all hospital acquired infections. In fact, 5% of patients having a surgical procedure will develop an infection.

Reducing the morbidity and mortality of wound infections is partially dependent on the timely recognition and appropriate treatment of the infection, which continues to be challenging despite the advancements in modern medicine. In 2015, the Institute of Medicine (TOM) underscored the need to reduce diagnostic error, challenging the medical community to urgently address the need to decrease the rate of delayed or inaccurate diagnoses. To address this pressing need, we sought to determine if thermography would be useful in assessing and confirming wound infections. LWIT can measure the radiant heat from the body surface and has been well accepted as a valuable adjunct to standard investigations in the early detection of inflammation and infection. Previous research has shown that a temperature difference between a chronically infected wound and normal tissue has a specific elevated thermal gradient range of ≧+3-4° C. In wounds where the infection was cleared but “healing” inflammation persisted, a thermal gradient of ≦+2° C. was noted. Thermography has been shown to be sensitive in detecting prosthetic knee infection, arthrosis, active rheumatoid arthritis, septic arthritis, sports injuries, osteomyelitis, inflammation, gangrene, abscesses, and sternal wound infections. These studies seem to suggest that thermography may play an important role in the early detection of wound and soft tissue infection to help improve patient outcomes, and ultimately to decrease health care costs.

### SUMMARY

The use of quantitative analysis of LWIT may be a useful tool to objectively and reliably identify a wound infection. The present invention comprises using LWIT to quantify the characteristic temperature changes associated with infected wounds. In the four specific examples presented herein, the method of LWIT of the present invention is used to accurately confirm infection, thus demonstrating the potential of this tool for the objective detection of wound infection.

The purpose of the observational retrospective case review series was to determine if wound infections are associated with specific temperature changes that can be detected and characterized by quantitative analysis of long wave infrared thermographic imagery. Secondarily, the researchers sought to evaluate temperature heterogeneity and wound measurements longitudinally as a means to evaluate wound healing. Previous studies have demonstrated that thermography has been safely used on humans and animals as a non-invasive method for measuring physiological and pathological changes in body surface temperature resulting from a number of conditions including infection and inflammation. In the present invention, an FDA-cleared Scout® dual imaging long wave infrared camera and digital camera is used to analyze images of wounds or areas of interest for four subjects with documented wound infections. Scout® is an FDA cleared non-contact and non-radiating combination digital camera and long wave infrared camera cleared for use in non-pregnant adults 18 years of age and older in the United States, and commercialized by WoundVision, LLC of Indianapolis, Indiana. In all four specific examples of using the present invention on a wound infection, quantitative analysis of long wave infrared images prior to treatment, demonstrated characteristic results confirming the presence of infection. In each of the cases, the first image obtained was prior to antibiotic therapy and revealed a maximum temperature difference between the wound and normal tissue (control) of +3.4 to +5.7° C. Post-treatment, the four subjects had maximum wound base temperature differences between the wound and healthy tissue ranging from +1.1° C. to +2.6° C. reflecting a narrower and lower temperature distribution. Additionally, for the three open wound trace perimeters, wound measurements decreased in size. For all four wounds, the thermally weighted Heterogeneity Index (a measure used to help quantify the alteration of thermogenesis associated with infection) showed that wound heterogeneity was significantly reduced following antibiotic treatment. Thus, the present invention demonstrates that quantitative analysis of LWIT images can be a useful tool in the identification and ongoing monitoring of wound infections.

In one exemplary embodiment, the present invention comprises a method of comparing the heterogeneity of a wound a two discrete times, said method comprising the steps of: providing an apparatus adapted to capture thermal images of biological material using LWIT; using said apparatus to capture a thermal image of a wound at a first discrete time; using said apparatus to capture a thermal image of said wound at a second discrete time; using a computer program to determine the weighted thermal Heterogeneity Index of said wound at said first time; using a computer program to determine the weighted thermal Heterogeneity Index of said wound at said second time; using a computer program to compare the weighted thermal Heterogeneity Index of said wound at said first time to the weighted thermal Heterogeneity Index of said wound at said second time.

In another exemplary embodiment, the present invention comprises a method of comparing the heterogeneity of a wound and the heterogeneity of a corresponding peripheral wound, said method comprising the steps of: providing an apparatus adapted to capture thermal images of biological material using long wave infrared imaging; using said apparatus to capture a thermal image of a wound; using said apparatus to capture a thermal image of a peripheral wound surrounding said wound; providing a computer program adapted to determine the weighted thermal Heterogeneity Index of said wound; using said computer program to determine the weighted thermal Heterogeneity Index of said peripheral wound; using a computer program to compare the weighted thermal Heterogeneity Index of said wound to the weighted thermal Heterogeneity Index of said peripheral wound.

### BRIEF DESCRIPTION OF THE DRAWINGS

The above-mentioned aspects and other characteristics and advantages of a system and/or method according to the present disclosure will become more apparent and will be better understood by reference to the following description of exemplary embodiments taken in conjunction with the accompanying drawings, wherein:

The various objects, features and advantages of the present invention will become fully appreciated as the same becomes better understood when considered in conjunction with the accompanying drawings. It is to be noted that the accompanying drawings are not necessarily drawn to scale or to the same scale; in particular, the scale of some of the elements of the drawings may be exaggerated to emphasize characteristics of the elements. Moreover, like reference characters designate the same, similar or corresponding parts throughout the several views, wherein:

FIG. 1 is a table showing demographics of evaluated subjects;

FIG. 2 is a chart showing a histogram of raw data for a wound site;

FIG. 3 is a chart showing a histogram of raw data for a control area;

FIG. 4 is a chart showing thermal distributions of wound sites compared to controls;

FIG. 5 is a table showing the Heterogeneity Index (HI) calculations for Subject 1 at discrete first and second imaging times;

FIG. 6 is a table showing the Heterogeneity Indices (HI) for each subject at the first and second imaging times;

FIG. 7 shows a visual image of the wound of Subject 1 at a first imaging time;

FIG. 8 shows a thermal image of the wound in FIG. 7;

FIG. 9 shows a visual image of the wound of Subject 1 at a second imaging time;

FIG. 10 shows a thermal image of the wound in FIG. 9;

FIG. 11 shows a visual image of the wound of Subject 2 at a first imaging time;

FIG. 12 shows a thermal image of the wound in FIG. 11;

FIG. 13 shows a visual image of the wound of Subject 2 at a second imaging time;

FIG. 14 shows a thermal image of the wound in FIG. 13;

FIG. 15 shows a visual image of the wound of Subject 3 at a first imaging time;

FIG. 16 shows a thermal image of the wound in FIG. 15;

FIG. 17 shows a visual image of the wound of Subject 3 at a second imaging time;

FIG. 18 shows a thermal image of the wound in FIG. 17;

FIG. 19 shows a visual image of the wound of Subject 4 at a first imaging time;

FIG. 20 shows a thermal image of the wound in FIG. 19;

FIG. 21 shows a visual image of the wound of Subject 4 at a second imaging time;

FIG. 22 shows a thermal image of the wound in FIG. 21;

FIG. 23 is a chart showing rTmax in the wound bed of each subject at the first and second imaging times; and

FIG. 24 is a chart showing wound trace perimeter lengths for each of three subjects at the first and second imaging times.

Corresponding reference characters indicated corresponding parts throughout the several views. Although the drawings represent embodiments of the disclosed apparatus, the drawings are not necessarily to scale or to the same scale and certain features may be exaggerated in order to better illustrate and explain the present disclosure.

### DETAILED DESCRIPTION OF EXEMPLARY EMBODIMENT(S)

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

Thus, all of the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The advantages of LWIT can be attributed to Planck's law and the second law of thermodynamics. Planck's law, simply stated, is that the intensity of radiation emitted from an object is a function of its temperature, wavelength, and emissivity. In theory, a perfect emitter, also known as a blackbody, radiates 100% of the electromagnetic energy as a function of temperature and wavelength.

Thermographic cameras generate images based on the amount of heat dissipated from the human skin surface in the form of electromagnetic radiation. Interestingly, human skin has an emissivity factor of 0.98 giving it a unique capability similar to the theoretical black body radiator, thus it is a nearly perfect emitter of infrared radiation at room temperature. Electromagnetic energy radiated from the skin surface corresponds to heat generation below the skin surface. The second law of thermal dynamics states that when bodies initially in thermal disequilibrium are put into contact with one another a new thermal equilibrium is achieved, which helps to explain the dissipation of energy in the form of heat. “Hot goes to cold,” and thus mean temperature (Tmean) serves as a summation indicator of the heat of contiguous areas. Thus, radiation from the skin is indicative of thermal activity of the skin itself and the heat generation that occurs below the skin surface. Tmean dilutes the measured temperature variability, effectively blending the high temperatures and low temperatures. To better document the temperature variability or heterogeneity in a wound, the method herein disclosed utilizes the maximum relative temperature differential (rTmax) in the contiguous areas forming the wound and the peripheral wound (the “periwound”) surrounding the wound itself (the “wound bed”). Relative to Tmean, the temperature differences within these areas represented by rTmax better characterize thermal dysregulation to help confirm the presence or absence of a critical wound infection. Previous researchers have characterized temperature ranges determined via LWIT as predicative of infection, in that there is a measurable and reproducible relative temperature range indicative of wound infection.

Importantly, the human skin emits electromagnetic wave radiation in the 2-20 μm range with an average peak of 9-10 μm. Because of the excellent emissivity of the skin in accordance with Plank's law, most of the emitted radiation from the human body is in the range of 8-15 μm. Topical skin temperature measurement means utilizing short and medium wave thermography are not sensitive enough to usefully measure these emissions in deep tissues, but LWIT can identify temperature changes therein. Furthermore, researchers have demonstrated that computational thermal modeling of thermographic results show that tissue ischemia and inflammation can be manifested as temperature decreases and increases.

Previous infrared thermography technology used single detector thermal cameras that had liquid cooled thermal cores, as compared to the above-mentioned Scout® device which employs a newer technology utilizing focal plane array detectors that can capture relatively longer wave infrared emissions in the 7-14 μm range. Because of their ability to detect electromagnetic radiation in the longer wave infrared spectrum, the focal plane array detectors are able to detect sub-epidermal thermal activity with more accuracy. In addition, the Scout° device has a high spatial resolution that provides its ability to discriminate between two contiguous areas, and a high thermal resolution that enables it to detect subtle temperature differences between two areas with a sensitivity of <50 mK. An embodiment of the Scout® device, an exemplary system including it, and an exemplary method utilizing them, is disclosed in U.S. Publication No. 2016/0027172 A1 entitled METHOD OF MONITORING THE STATUS OF A WOUND, the entire disclosure of which is incorporated herein by reference.

Results obtained by a method according to the present disclosure are objective, reproducible, and cost effective, and increase diagnostic accuracy in detecting and monitoring wound infections. As herein disclosed, analyzing sub-epidermal heat generation activity according to the present invention, with a device such as the Scout® camera device and its software, demonstrably supports a clinician's ability to confirm the presence of infection with greater accuracy than achieved heretofore.

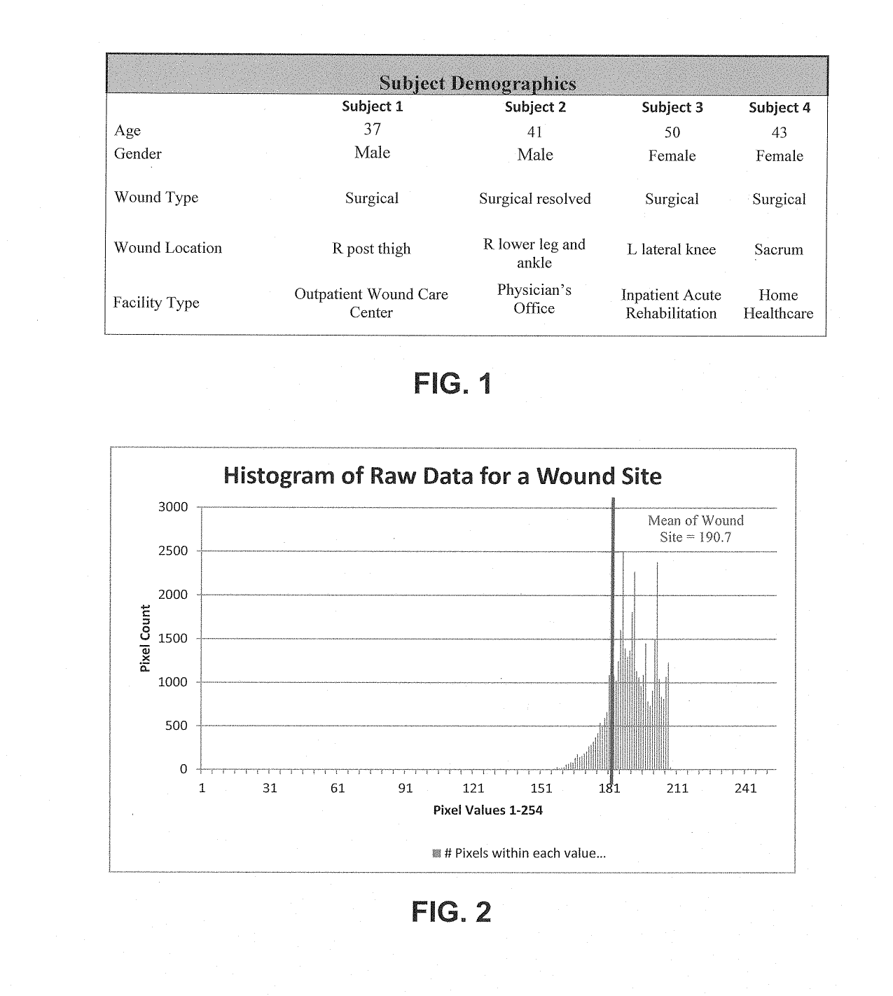

The present disclosure reviews four case studies of subjects described in FIG. 1, each with wound infections. This disclosure presents quantitative analysis of data obtained utilizing LWIT that confirmed the presence of infection using two criteria. The first criterion was the temperature difference between a normal tissue control site and the wound site, which includes the wound itself and the periwound. The second criterion was an evaluation of thermal heterogeneity as a measure of the alteration of thermogenesis that may be associated with infection. The disclosure demonstrates the use of quantitative analysis of LWIT data to provide time sensitive, non-invasive and cost effective objective support for the early identification of wound infections, thereby reducing diagnostic error and its attendant consequences. The study demonstrates the utility of the present invention in allowing clinicians to accurately quantify and monitor treatment of infections in wounds.

LWIT is able to detect the characteristic temperature range of infected wounds in a number of clinical settings and thus confirm the presence of infection to improve patient care in any setting. Infected wounds exhibit an increased number of temperature values, heterogeneity, and an index was created to determine if the temperature heterogeneity decreased and if wound size decreased during the course of wound treatment. In accordance with the present method, a device such as the Scout® device, used with its LWIT measurement software, allows clinicians to accurately quantify and monitor the treatment of infections in wounds.

The digital camera of the Scout® device captures the light wavelengths from the electromagnetic spectrum that is visible to the human eye. The thermal camera of the Scout® device captures long wave infrared thermal electromagnetic spectrum wavelengths not visible to the human eye. The paired images are then evaluated utilizing proprietary software to measure the periwound length and width, and temperature differentials between the wound, periwound, and wound site as compared with a control.

Previously obtained de-identified images of the subjects were captured in multiple settings including home health care, out-patient wound care clinic, acute rehabilitation hospital, and a wound care physician's office with the Scout® system. There was no anticipated benefit or risk to the subjects who participated in this study, and the study was conducted in compliance with the protocol, the FDA's Good Clinical Practice Guidelines, Ethical Guidelines of the 1975 Declaration of Helsinski and all applicable regulatory requirements.

In this case series, the subject patients were selected if they had (1) consent for imaging with the device; (2) they had a wound infection and were treated with antibiotics; and (3) were being treated in one of four clinical settings. These settings included a home health care setting, an out-patient wound care clinic within an acute care hospital, an acute rehabilitation hospital, and a private physician's office from April 2011 to May 2015. The images were included for review if there was a temperature differential of ≧+2° C. The four subjects included in the case series review had elevated temperature differential and clinical diagnosis of infection. Additionally, the inclusion criteria required that thermal imaging utilizing the Scout® system be obtained during at least two time periods including the discrete imaging times that the patient was diagnosed with infection and when treatment was completed or while completing antibiotic therapy. All of the patients signed an informed consent to be imaged and allow the clinical data to be collected for research purposes. The images and clinical data collected were de-identified and submitted to the Indiana University Investigational Review Board (IRB) for approval. The study images and associated clinical data were deemed exempt by the IRB. Subject demographics are shown in FIG. 1.

Each clinician utilizing the Scout® system was trained on the proper use of the Scout® system by qualified WoundVision staff members. Adhering to the manufacturer recommendations, images from the study were taken at a 90° angle to the skin surface and 46 cm away from the wound site. Wound dressings and clothing were removed and tissue proximal to the wound was exposed and acclimated to ambient room temperature for 5 minutes prior to imaging. In all four settings, the room temperature ranged from 18-35° C. and was free from external heating or cooling effects including direct sunlight, fans or direct heating devices such as heating pads or space heaters.

The Scout® system includes proprietary software that allows for accurate measurement of wounds using visual imaging, including standard length by width in centimeters, volume (when depth is manually recorded), surface area by pixel count included within a wound trace perimeter, and perimeter trace length of a wound or area of interest. Wound trace perimeter length was used as the primary basis for size measurement in the case report series, since studies show it is more reliable and accurate in following wound progression or regression as compared to wound trace area, though certain embodiments of a method according to the present invention could additionally or alternatively, as done herein, utilize wound trace area measurements.

To obtain the wound perimeter measurements, the Scout® device utilized was held at a distance of 46 cm away from the subject established by the convergence of two lasers when the camera is at the prescribed distance, roughly 90° to the body surface. The size of the wound is accurately calculated with the Scout® system software. A test target size of 1.5″×1.5″ yielding a test target area of 2.25 in² or 14.52 cm² was obtained utilizing the calibration technique of the visual image camera. For a visual image taken at 46 cm from the target (when the lasers converge), there would be approximately 40 pixels per inch and so there would be approximately 60 pixels in 1.5 inches. The area of the test target obtained from the visual image is 60 pixels×60 pixels, which is equal to 3600 pixels. The test target area of 14.52 cm² was divided by the 3600 pixels, which resulted in a single pixel surface area of 0.004 cm². In performing a wound trace perimeter, the software identifies the number of pixels contained within that trace, and determines the wound trace area. For example, if 7200 pixels are contained within the wound trace perimeter, the wound trace surface area is 28.8 cm² (7200×0.004).

In accordance with the present invention, the wound bed and periwound temperatures are evaluated utilizing pixel values, thereby providing an adjunctive tool to help reveal and quantify thermal temperature aberrancies that may indicate a disease process not seen by the eyes.

As noted above, the actual wavelengths discernable utilizing the Scout® device are between 7 μm and 14 μm. This range matches the human body's most efficient electromagnetic spectrum radiation of heat. In each imaging session, a thermal image and a visual image are taken simultaneously. The initial data of the thermal image is captured in the native gray scale by evaluating the number of pixels within each shade of 1 to 254 gray scale values, with relatively higher “pixel values” (not the number of pixels) indicating relatively warmer temperatures. In the study, the thermal camera was purposefully calibrated in the range of 20° C. to 40° C. to match the temperature range of a living human organism, and providing a 20° C. range corresponding to the 254 pixel values. Dividing the 254 pixel values by 20° C. yields 12.7 pixels per degree Celsius. Overlaying a wound trace perimeter from the visual image onto the corresponding thermal image allows the Scout® system to identify the number of each pixel value contained within the tracing. In thermal images, higher pixel values appear as whiter areas, and represent warmer temperatures; lower pixel values appear as blacker areas, and represent cooler temperatures. FIG. 2 shows the pixel count associated with each pixel value in a thermal image of an exemplary wound site.

Respective to each of the four subjects, a control area was identified near the wound. Theoretically, the reason a control area is selected is because the control and the wound or area of interest are affected similarly by the environment and intrinsic host factors, so using the relative value should mitigate the effects of those factors that may influence skin surface temperature anomalies.

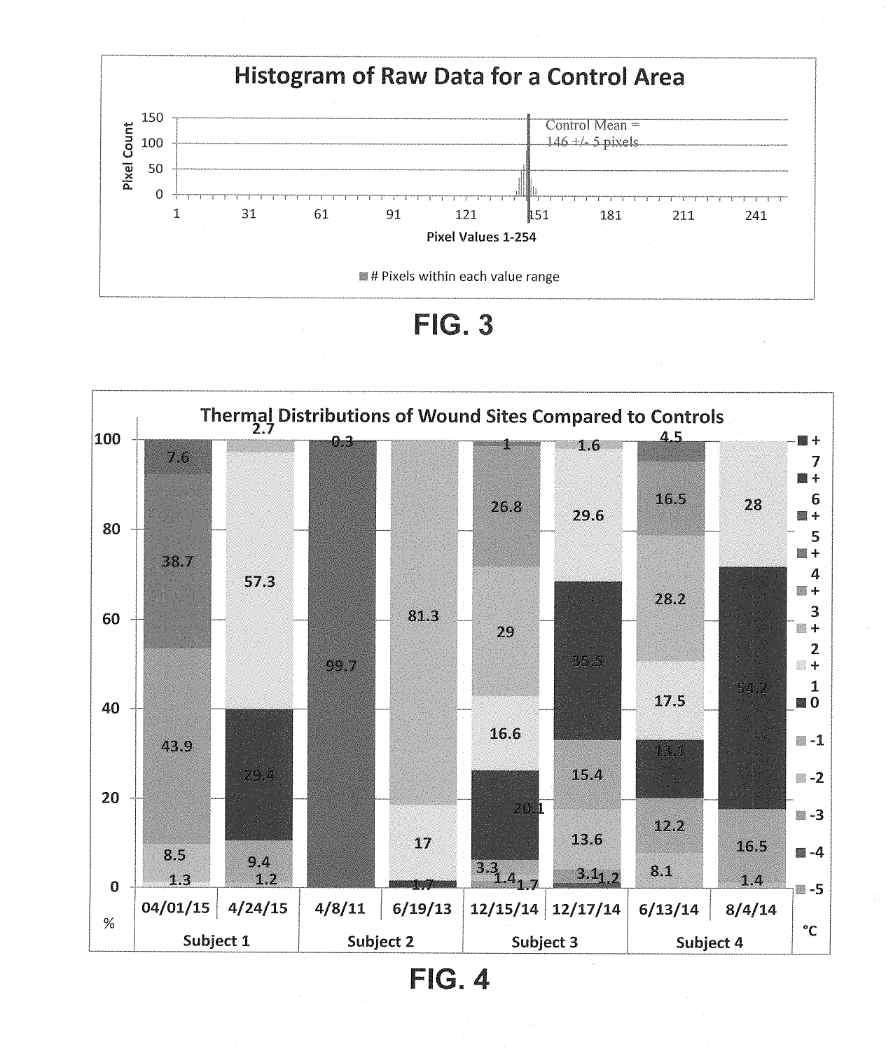

A control area was defined as an area of unaffected tissue proximal to the wound site that fulfills two criteria: (1) the control area has a temperature variation within itself of no more than 1° C.; and (2) the control area is not over a bony prominence, a large blood vessel or visible wound/skin anomaly. In the method embodiment utilized in the study, the control area was a circle comprised of 437 pixels in size. Referring to FIG. 3, the mean of the pixel values contained in the control area of each particular subject, plus and minus 0.5° C., were standardized and converted to a 0° C. baseline representing the temperature of the control. The method herein disclosed involves the determination and evaluation of relative temperatures (i.e., relative to the control) rather than of absolute temperatures. Wound temperatures and their variances are to be compared and assessed relative to this control baseline, that is, the control is compared with temperatures, represented by pixel values, in the wound bed, periwound, and wound site to obtain the temperature gradiency and thermal distribution.

Respective to the pixels identified within the wound trace perimeter, the number of pixels that fall within each shade of gray is calculated and is placed into a “pixel bucket” which is made up of a 1° C. increment. Each 1° C. increment is made up of 0.5° C. below and above each number. For example 0° C., which represents the baseline defined by the mean temperature of the control area, is made up of all pixel values of the wound trace, relative to the control, falling in the range of −0.5° C. to +0.5° C. Similarly, the +1° C. increment is made up of all pixel values of the wound trace, relative to the control, ranging from +0.5° C. to +1.5° C., and so on. Once compared with the control area baseline, the data obtained from the wound site is then represented in the form of different pixel value percentages on the thermal distribution chart of FIG. 4, which includes data pertaining to each of the four subjects of the study at their respective first and second imaging times.

In certain embodiments of the method, maximum relative temperature differentials (rTmax) are identified/reported and utilized to avoid the dilutional effect of hot and cold tissues in close proximity. The rTmax values also serve to assist in confirming the presence or absence of infection.

In addition to the temperature differential, in certain embodiments of the method a weighted thermal Heterogeneity Index (HI) is calculated based on the thermal images obtained with the Scout® system. The HI is a continuous scale that represents the percentage of pixel values contained in each degree Celsius present in the wound, periwound, or wound site. Evaluation of the HI may help to identify wounds or other areas of interest (AOIs) that are at greater risk for infection and thus impaired wound healing.

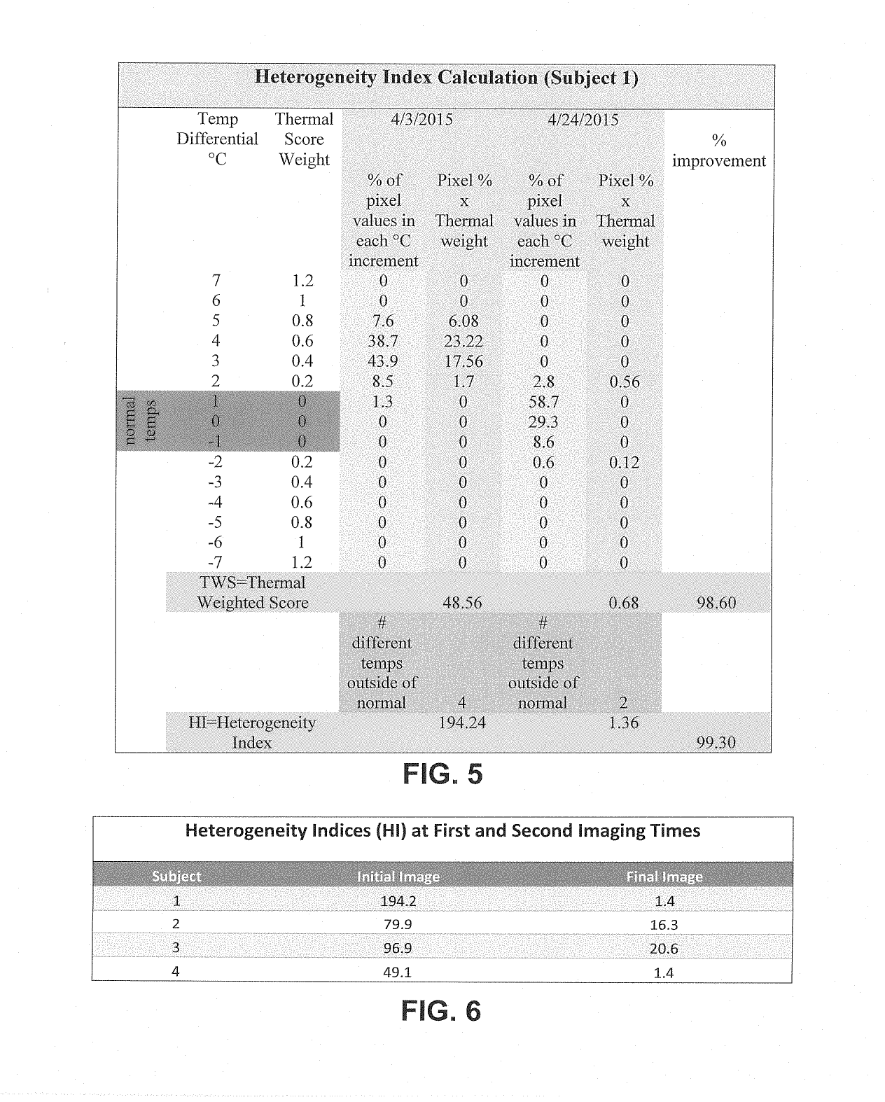

The HI is comprised of two weighted scores, a thermal weighted score (TWS) and a heterogeneity weighted score (HWS). As noted above, the Scout® system takes a thermal image and a digital image of the wound and records the percentage of pixel values in each 1° C. increment present in the wound or AOI. Referring to FIG. 5, which relates to the wound site of Subject 1, thermal score weights of 0 to 1.2 are assigned for each 1° C. increment/decrement from the control baseline, from 0° C. to 7° C., with greater temperature differentials being assigned a higher score, for they indicate a more dysregulated thermogenesis and greater deviation from normal heat generation. For each 1° C. increment/decrement from the control baseline, the percent of pixel values contained in the wound base, periwound or wound site are produced as output and reported by the Scout® system. For example, the imaging performed on Apr. 3, 2015 indicates that a subarea defining 43.9% of an entire wound site shows pixel values that are 3° C. higher than the control area baseline, whereby Scout® system outputs 43.9% in the +3° C. range. As shown in FIG. 5, a thermal score weight of 0.4 is assigned to a 3° C. increment/decrement, which in this case is multiplied by 43.9 to obtain the thermal weighted value of 17.56. Each 1° C. increment/decrement is handled in the same manner. All thermal weighted values are added together to obtain the TWS, which in this case is 48.56 for the Apr. 3, 2015 imaging, according to the following formula:

Σ(%pixel values of subarea*temperature weight)=TWS

HWS is the number of wound temperature values that are outside the range of normal temperatures at the respective imaging time, defined in this example as the control baseline of 0° C. +/−1° C. Respective to the Apr. 3, 2015 imaging data shown in FIG. 5, there are four (4) wound temperature values outside the range of normal temperatures, which is from −1° C. to +1° C. HWS is thus 4.

The Heterogeneity Index is the product of TWS and HWS, thus:

TWS*HWS=HI

HI is calculated for each imaging session for each respective subject. Wounds with a higher number of different temperatures (i.e., higher HWS) have more aberrant temperature differentials, and thus a higher HI score.

Finally, referring to still to FIG. 5, the HI percentage of improvement or degradation is calculated by comparing the initial image HI (at a first time, e.g., Apr. 3, 2015) to the subsequent or final image HI (at a second time, e.g., Apr. 24, 2015).

In the study, an HI is calculated for each of the four subject patients, for each of the respective subject's two imaging sessions, with the results shown in FIG. 6. Between two HI values of a subject's wound based on images obtained at first and second times, the percentage change in HI is calculated as follows:

(Initial image HI−Final image HI)/Initial image HI*100=HI percentage change Positive values of HI percentage change indicate reductions in HI and such a reduction may be considered an improvement of the wound condition at the second imaging time relative to the wound condition at the first imaging time, whereas negative values of HI percentage change indicate increases in HI and such an increase may be considered a degradation of the wound condition at the second imaging time relative to the wound condition at the first imaging time. Returning to the example reflected in FIG. 5, HI improved by 99.30% between the first imaging time (Apr. 3, 2015) and the second imaging time (Apr. 24, 2015).

### EXAMPLES

Subject 1:

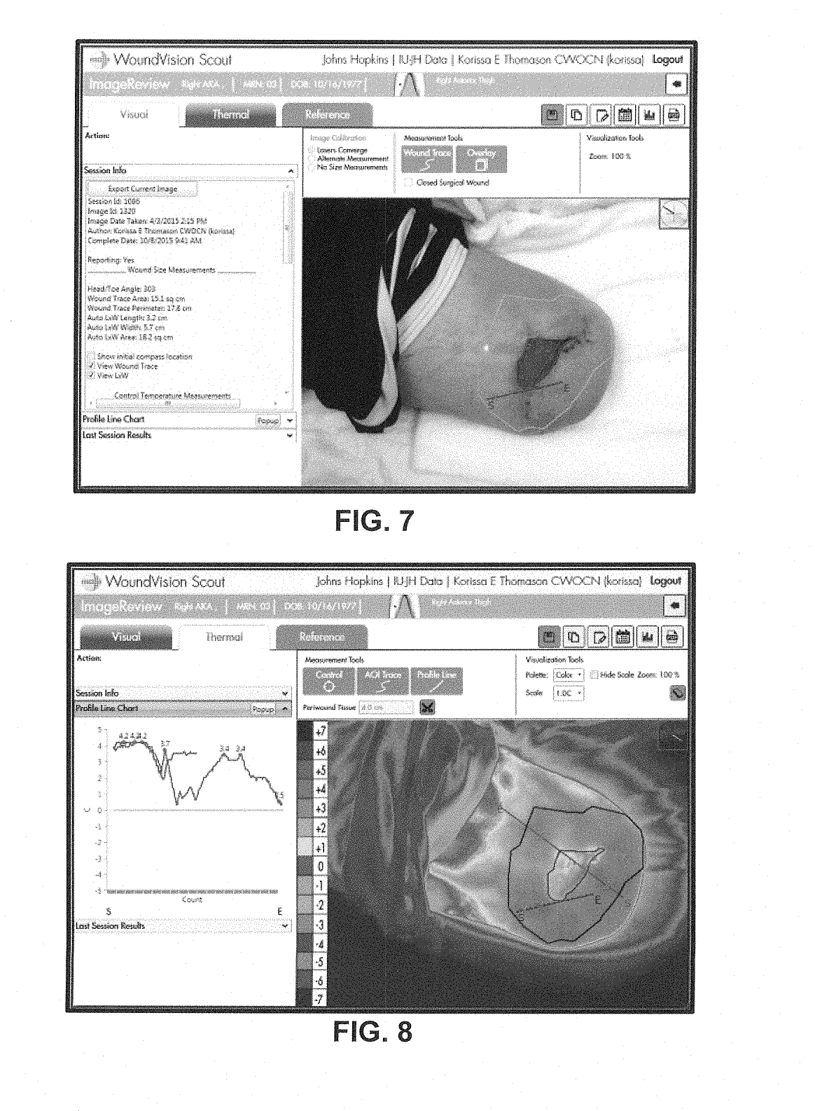

A 37 year old male was struck by a car on Feb. 5, 2015 resulting in a right above the knee amputation. The patient was being followed in an outpatient wound clinic for an open wound of the distal stump flap. When seen on Mar. 19, 2015, the wound showed an increase in warmth, induration, erythema, pain, and purulent drainage of the distal stump. A surgical incision and drainage of a wound abscess with wound culture was performed. He was empirically started on Cephalexin for ten days. The wound culture returned showing 4+growth of Bacteriodes fragilis, Prevotella species, Streptococcus viridans and 3+positive for Enterococcus. The Cephalexin regimen was continued through Mar. 28, 2015. Upon return to the clinic on Apr. 3, 2015, the patient had increasing erythema and warmth of the distal stump in the periwound at 6 o'clock, along with purulent drainage and foul odor (See FIG. 7). The patient was subsequently placed back on Cephalexin for another 10 days. On Apr. 24, 2015, the patient had completed his antibiotics and was deemed free of infection.

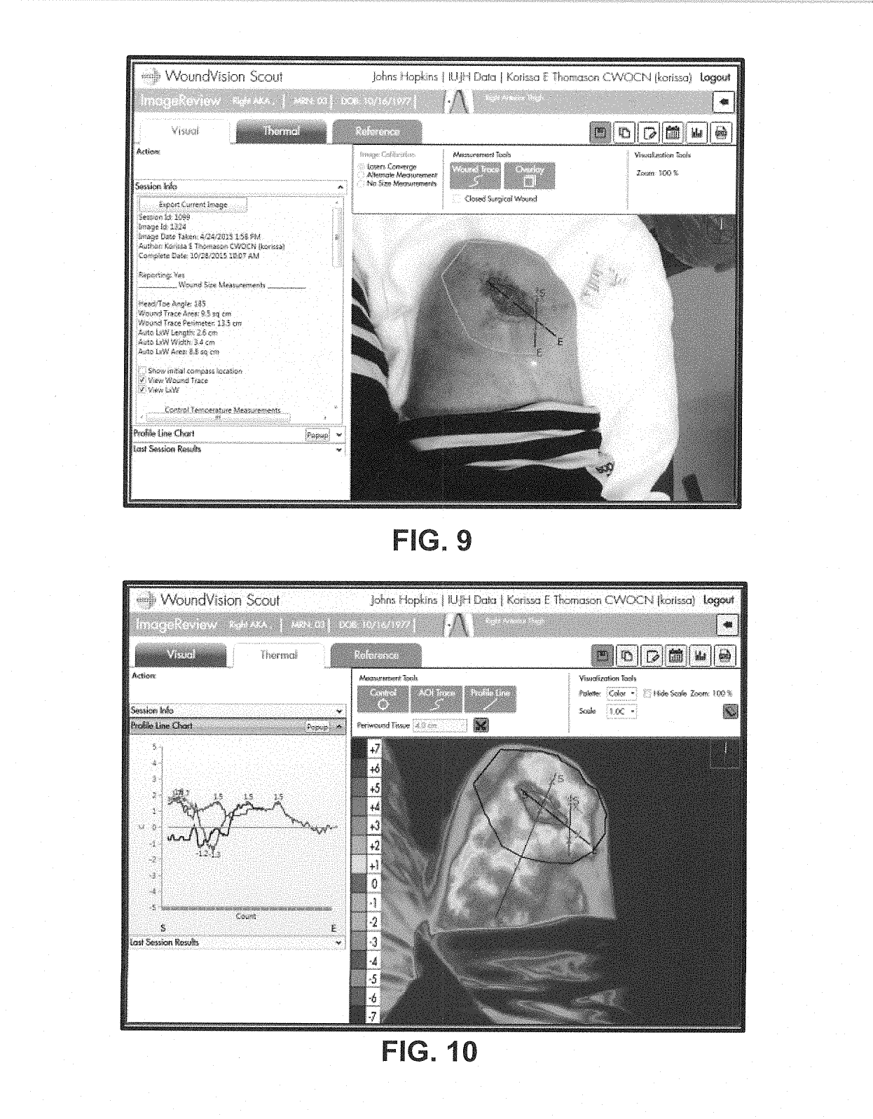

Thermal imaging at the initial visit revealed the temperature differential of the wound base +5° C. and periwound temperature differential to be +4.9° C. (See FIGS. 7 and 8). Final thermal imaging showed the periwound temperature differential to be +1.5° C. and the wound bed differential was now −1.3° C. (See FIGS. 9 and 10). Wound trace perimeter length decreased from 17.8 cm at the time of infection (the first imaging time) to 13.5 cm following resolution of the infection (at the second imaging time), indicating a 24.2% improvement. Wound trace area correspondingly decreased from 15.1 cm² to 9.5 cm², a 37.1% improvement. With respect to the heterogeneity, there were five different 1° C. temperature ranges in both the pre and post-antibiotic image sessions, demonstrating a HI of 194.2 initially and 1.4 after antibiotic treatment, demonstrating a 99.3% reduction (improvement).

Subject 2:

A 41 year old male fell off a roof in January 2010 fracturing his right tibia, fibula and talus bones. Open surgical reduction with plating was performed. His post-operative course was complicated by severe unrelenting pain which prevented him from returning to work. A triphasic bone scan was obtained on Jul. 20, 2010 and revealed increased activity in the distal tibia, fibula and prominently in the dome of the talus. Differential diagnosis included avascular necrosis and infection but infection was not pursued because no visible signs were present. He sought a second opinion with a wound care physician on Aug. 20, 2010. Physical examination at that visit revealed well healed surgical wounds and 1-2+ edema of the right ankle without erythema. An MRI of the right lower leg and ankle and C-reactive protein (CRP) were obtained. CRP returned elevated at 14.6 mg/L (0.0-4.9). MRI revealed marked bone marrow edema involving distal tibia, distal fibula and talus. Differential diagnosis included osseous stress reaction, reflex sympathetic dystrophy and osteomyelitis. Conservative treatment was continued as the right ankle edema and pain seemed to be slowly improving. On Apr. 8, 2011, the patient returned to clinic complaining of severe pain in the right leg and chills. The patient was diagnosed with osteomyelitis and started on Levofloxacin for 8 weeks. Subsequently, the patient went on to resolution of pain completely and was able to return to work.

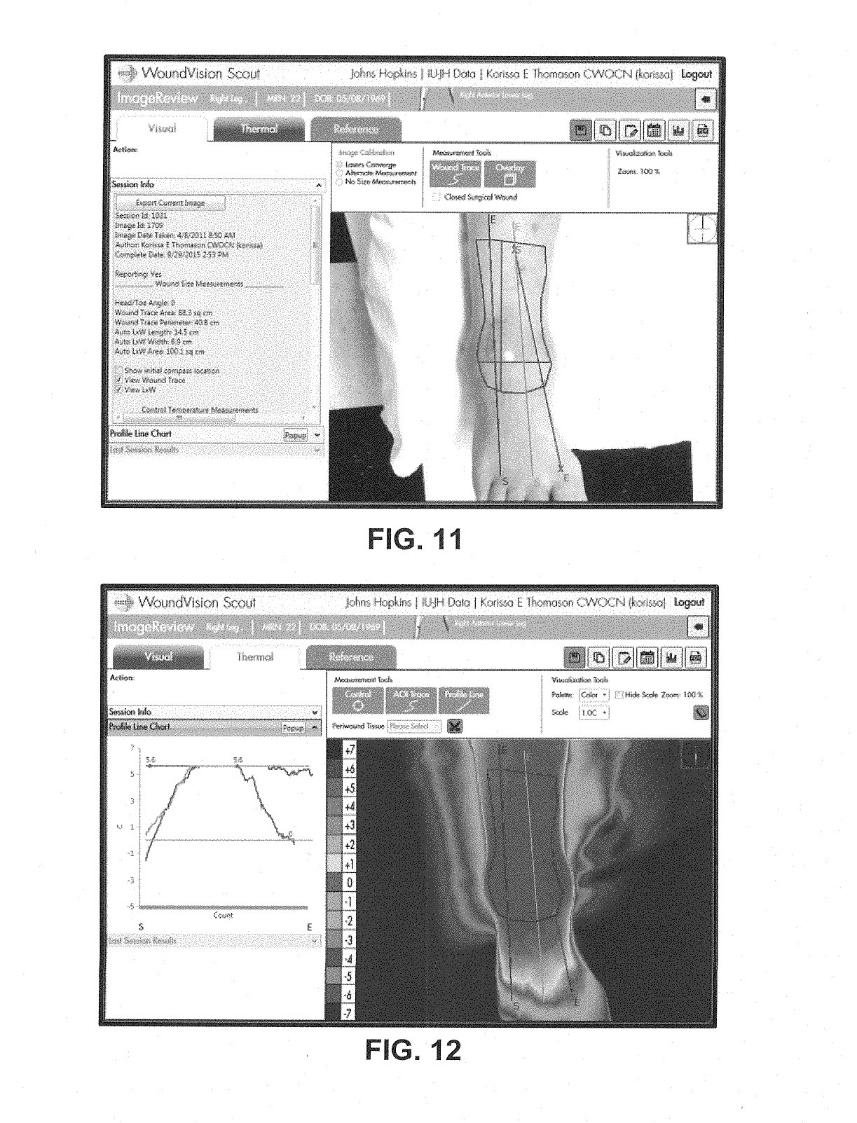

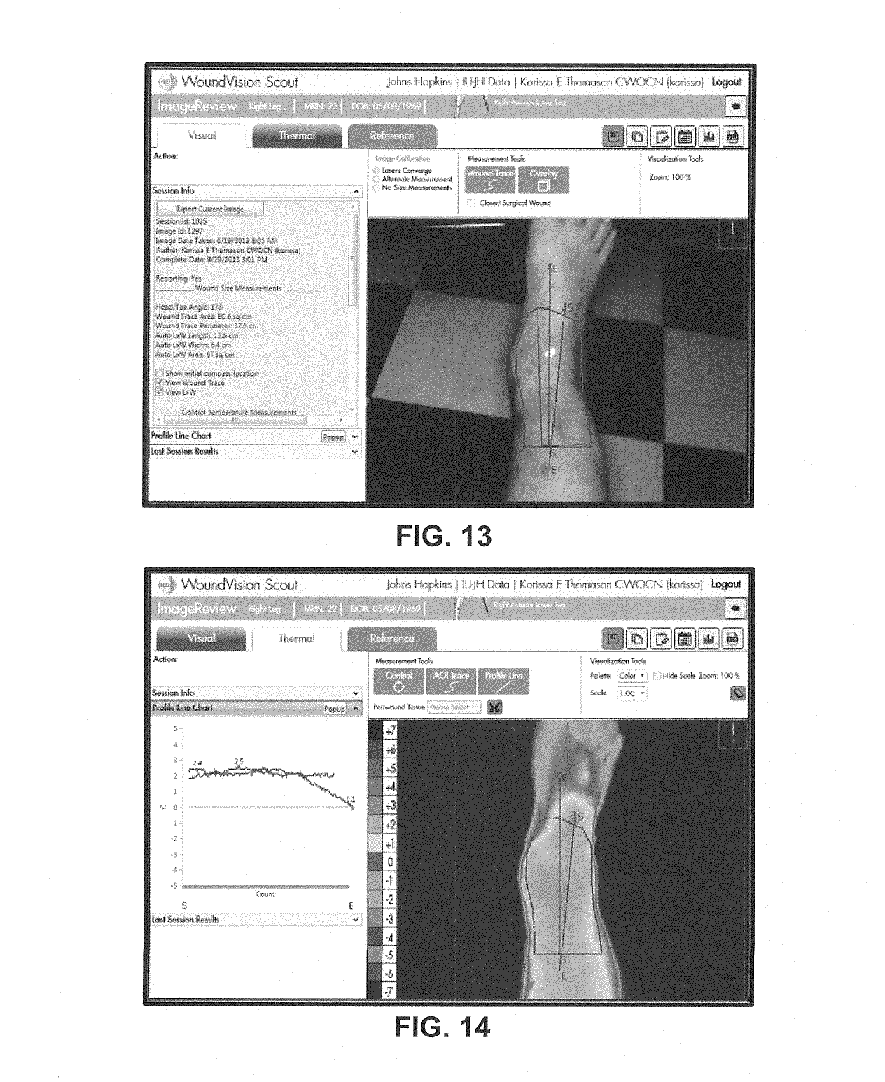

Initial Scout® imaging confirmed a temperature differential of the right lower extremity to be +5.6° C. (FIGS. 11 and 12). Follow up Scout® imaging on Jun. 19, 2013 revealed the temperature differential of the right lower leg to be +3.4° C. (FIGS. 13 and 14). With respect to the heterogeneity, there were two different 1° C. temperature ranges in the pre-antibiotic image, and three in the post-antibiotic image session, demonstrating a HI of 79.9 initially and 16.3 after antibiotic treatment demonstrating a reduction of 93.2%.

Subject 3:

A 50 year old female with multiple sclerosis and diabetes mellitus type II presented to an acute care hospital on Nov. 30, 2014 with complaints of left knee pain, nausea, vomiting, excessive thirst, shortness of breath and elevated blood sugar of 599. The patient was diagnosed with a left knee bacterial bursitis which was believed to the source of sepsis, diabetic ketoacidosis and acute renal insufficiency. The patient underwent a left knee arthrotomy incision and drainage of an abscess on Dec. 1, 2014. Cultures grew out MRSA and she was started on Vancomycin. The surgical wound was left open and the patient was discharged to an acute rehabilitation hospital on Dec. 13, 2014.

The wound care team was consulted on Dec. 15, 2014 and they felt the wound was still infected so the Vancomycin was continued. Two days later the patient was seen by the wound care team who deemed that the infection was resolving based on the appearance of the wound and also had an improvement in symptoms. Importantly, the wound bed slough had decreased.

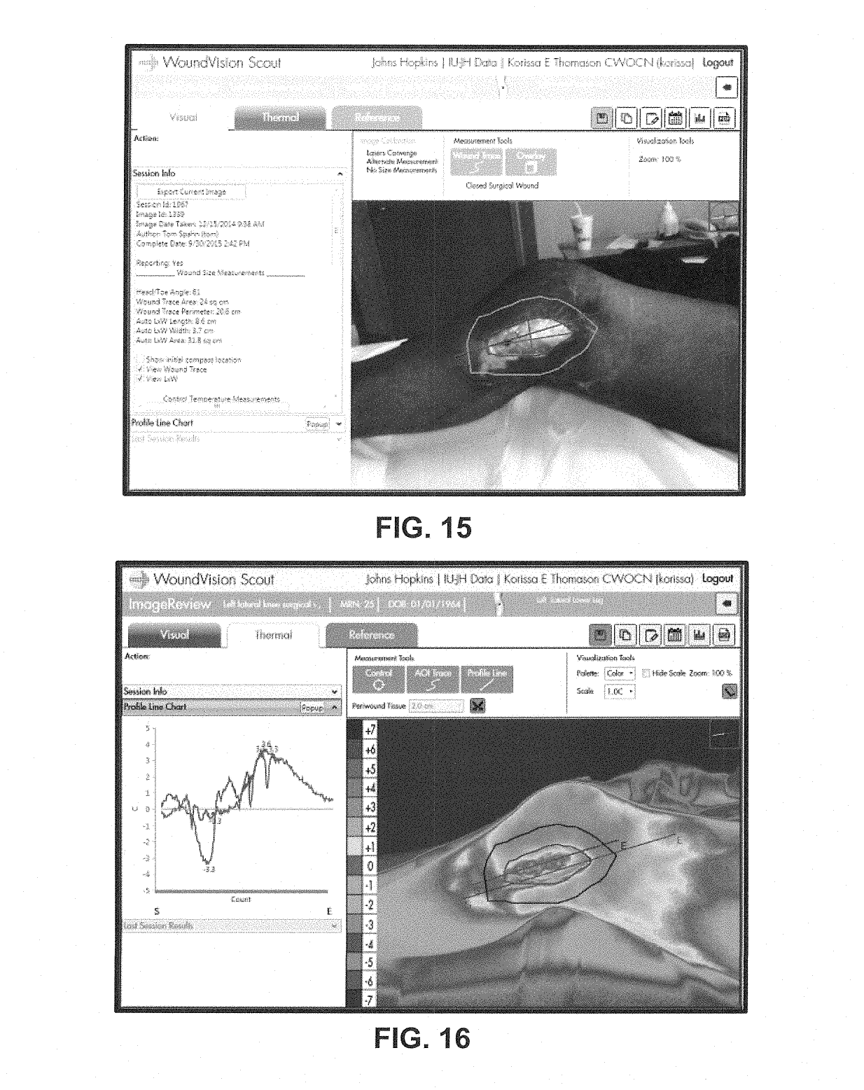

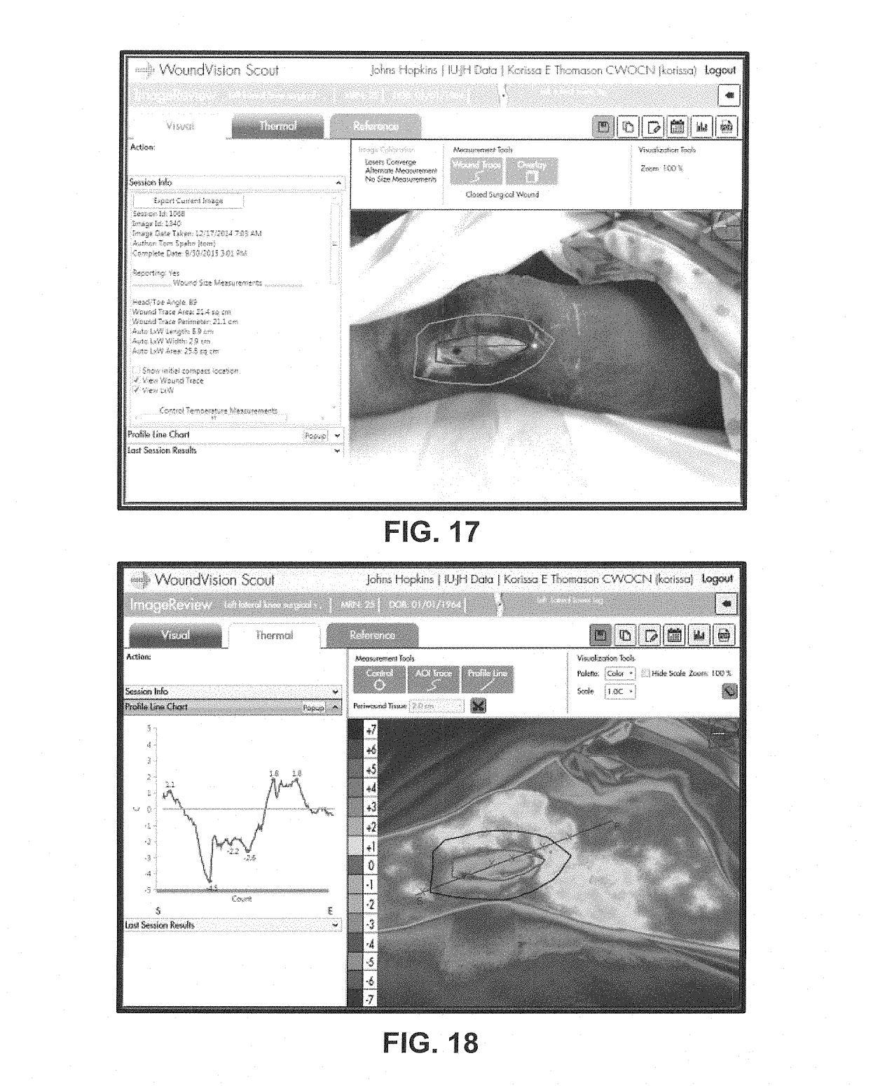

Referring to FIGS. 15 and 16, the initial thermal image (Dec. 15, 2014) revealed a temperature differential of the inferior wound bed to be −0.3° C. the cephalic wound bed had a temperature differential of +3.3° C. and the periwound temperature differential was +3.6° C. Referring to FIGS. 17 and 18, the final thermal image revealed the wound bed temperature differential to be 0° C. to −4.5° C. The periwound temperature differential had decreased to +1.8° C. While the wound trace perimeter length increased slightly (2%) from 20.6 cm to 21.1 cm, the wound trace area decreased from 24 cm² to 21.4 cm², an improvement of 10.8% after two days of antibiotics. The patient signed out against medical advice on Dec. 30, 2014, so no further images were obtained. There were eight different 1° C. temperature ranges in the pre-antibiotic image and seven in the follow up image session (while still on antibiotics). The HI was 96.9 initially and 20.6 after continuation of the antibiotic, showing that although antibiotics weren't complete, the HI decreased by 78.7%.

Subject 4:

A 43 year old female was evaluated by a home health care agency clinician with history of paraplegia due to spina bifida as well as a pressure ulcer that was previously closed surgically but dehisced. The subject was evaluated by her wound care physician and on Jun. 16, 2014 was admitted to an acute care hospital for a wound infection, PICC line placement and wound debridement. The subject was diagnosed with an abscess and cellulitis. The abscess was drained and the patient was started on vancomycin and ertapenem. The patient was discharged back home on Jun. 18, 2014 for continued home health care. There she continued to improve and on Aug. 4, 2014 and she was deemed free of infection by her physician.

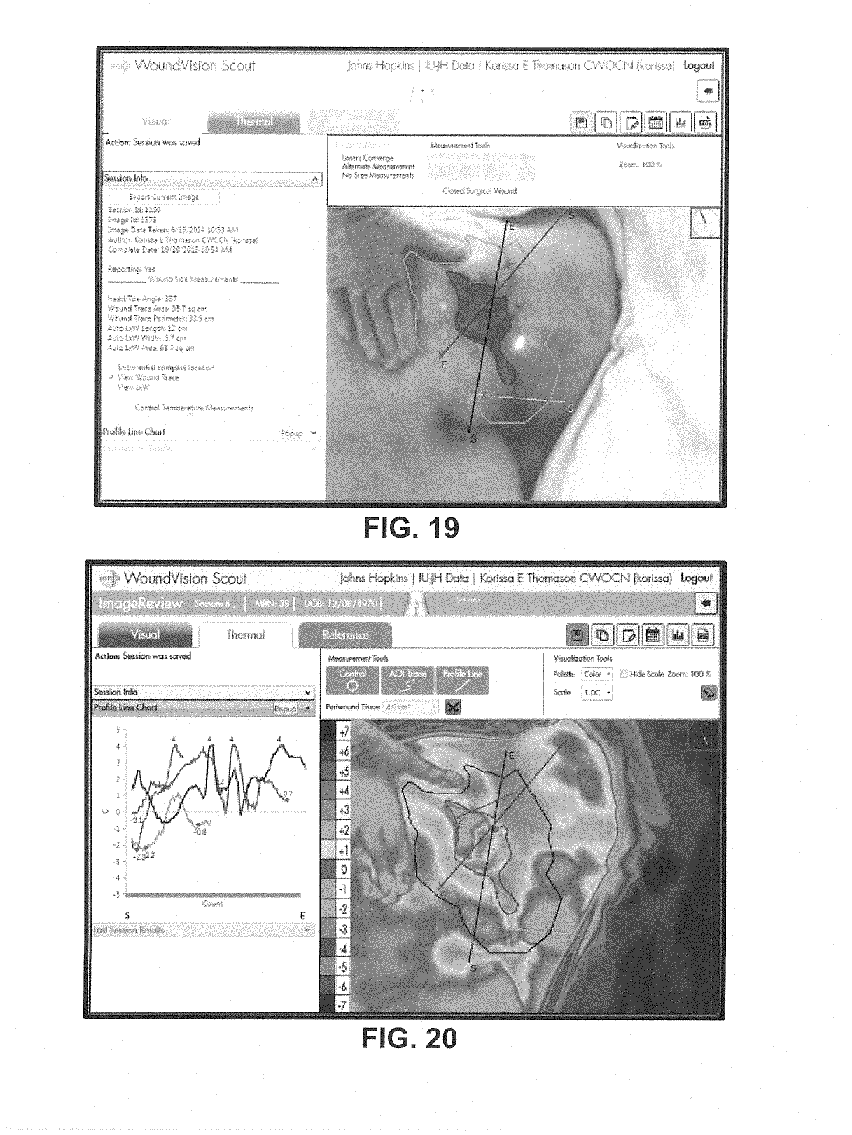

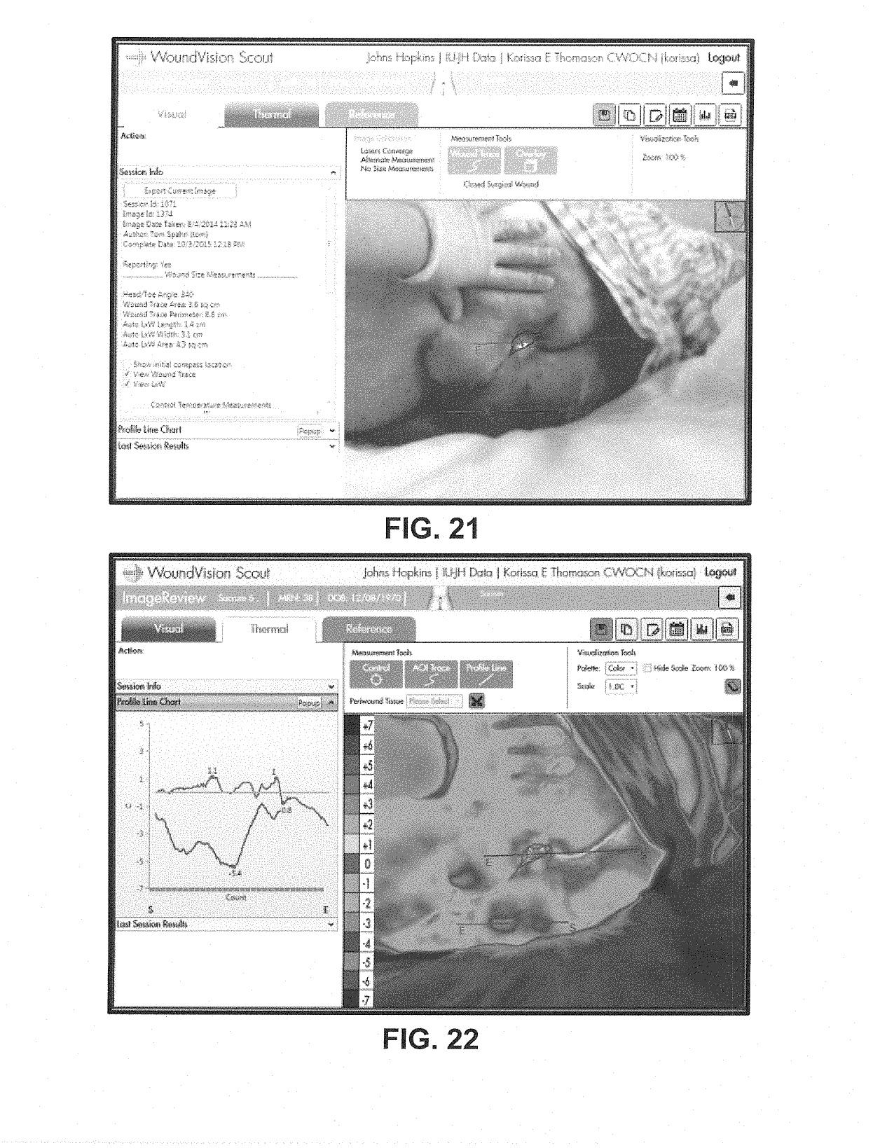

Initial imaging on Jun. 13, 2014 (FIGS. 19 and 20) showed the patient had a temperature differential in the central superior wound opening of −2.3° C., with the lower two thirds temperature differential markedly warmer at +4° C. Final imaging on Aug. 4, 2014 (FIGS. 21 and 22) showed the wound base temperature ranged from −0.8° C. colder to +1.2° C. warmer than the control, and the post incision and drainage region temperature differential was now only +1° C. The wound trace perimeter length decreased from 33.5 cm to 8.8 cm, indicating a 73.8% improvement. Wound trace area correspondingly decreased from 35.7 cm² to 3.6 cm², a reduction of 89.9%. There were seven different 1° C. temperature ranges in the pre-antibiotic image and four in the post-antibiotic image session. The HI was 49.1 initially and 1.4 after antibiotic treatment which was improved by 97.1%.

Discussion:

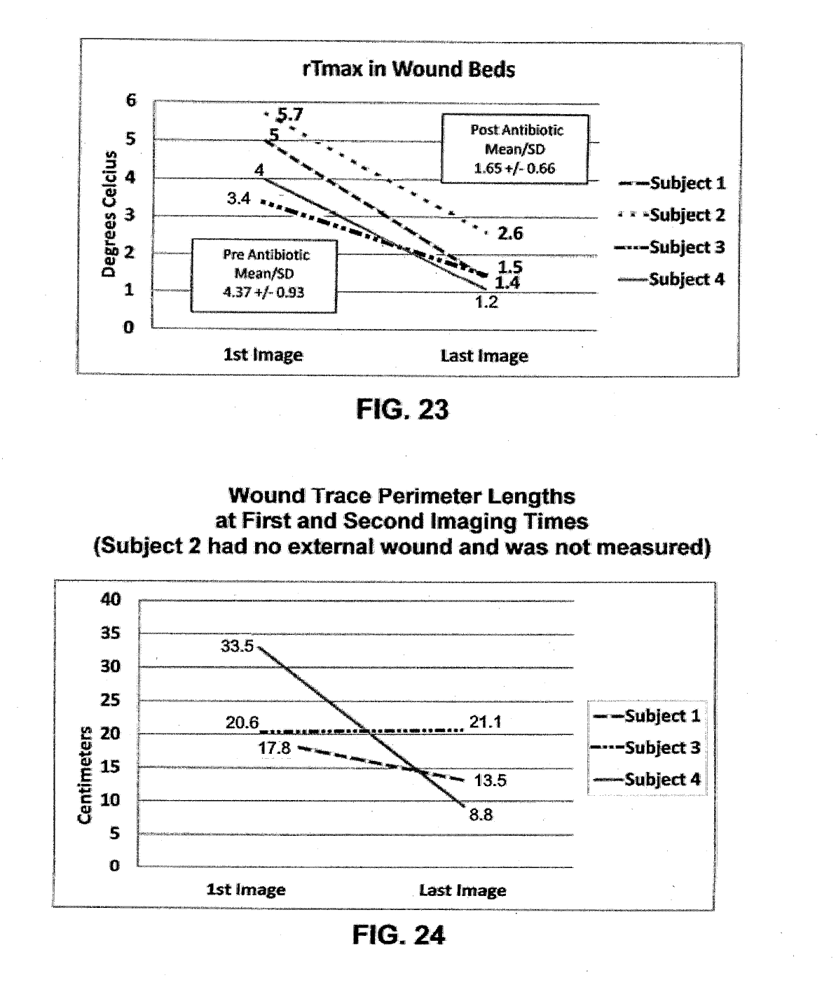

With reference to FIG. 23, the temperature differential (rTmax) indicative of wound infection in this case series, at the first imaging times, ranged from +3.4° C. to +5.7° C. with a Tmean of +4.34° C. (standard deviation +1-0.93° C.). Subject 2, diagnosed with osteomyelitis, had the most significant pre-antibiotic rTmax of +5.7° C. (FIG. 4).

rTmax in post-antibiotic images of the subjects (at the second imaging times) ranged from +1.2° C. to +2.6° C. with mean of +1.65° C. (standard deviation +/−0.66° C.). Subject 2 had a +2.6° C. rTmax, which may indicate a post infection healing inflammatory state since the temperature range for inflammation is postulated to be +1° C. to <+3° C. In this case series, subjects with maximum wound bed temperatures of ≧+3° C. had clinical infection, which is in agreement with prior studies.

In consideration of temperature heterogeneity, all subjects had a significant shift toward becoming less heterogeneous, as reflected in their reduced HIs, after being treated for a wound infection. Referring again to FIG. 6, the HI at the respective first imaging times ranged from 49.1 to 194.2, and the HI at the respective second imaging times ranged from 1.4 to 20.6. However, Subject 3 who had a final image (second imaging time) HI of 20.6 had not completed antibiotics as the other subjects had. After removing that outlier, the second time range is 1.4 to 16.3.

Finally, when looking at wound measurements, it was found that in the three cases with open wounds (Subjects 1, 3 and 4), treating the infection facilitated a healing trajectory in that all of the wounds experienced a reduction in either wound trace area and/or wound trace perimeter length between the respective first and second imaging times, as indicated in FIG. 24. Subject 2 had no open wound, so measurements were not possible. Subject 4 had the sharpest decline in wound trace perimeter length, which it dropped from 33.5 cm to 8.8 cm which represented a 73.7% reduction in wound trace perimeter length. Subject 1 had a reduction in wound trace perimeter length of 24.2%. Subject 3 had the lowest level of wound size change, which saw a slight wound trace perimeter length increase from 20.6 cm to 21.1 cm, but a wound trace area reduction from 24 cm² to 21.4 cm², a 10.8% improvement, after only two days. Another interesting finding is that when comparing wound improvement as measured by size reduction percentage and HI change (FIG. 6) the wounds follow a similar pattern, indicating a pattern of wound improvement that follows with the HI.

The results of this observational case series demonstrate the ability to detect wound infections in a variety of clinical settings and confirm that wounds with a temperature differential of ≧+3° C. and significant temperature heterogeneity are likely to be infected. Wounds showing a temperature differential of +1° C. to <+3° C. and a narrower temperature heterogeneity are likely to indicate a healthy post infection “healing” inflammatory state. Previously published studies are in agreement with the temperature differential of +1° C. to <+3° C. for inflammation with one caveat. In total knee and total hip implant surgeries, the elevated post-operative temperature gradient can reach >+3° C. In the immediate post-operative period for orthopedic implant patients, there is a well-defined temperature differential curve. The elevated temperature differential reaches a peak around day 3 to 4 and slowly declines to reach a normal temperature in approximately 90 days. Deviation from the normal healing curve should cause concern for a possible infection.

With respect to the determining a temperature range to indicate risk of wound infection, we found that in the first images at diagnosis of infection, the rTmax ranged from +3.4° C. to +5.7° C. The greatest rTmax, +5.7° C. was observed in the subject with osteomyelitis (Subject 2). Post-antibiotic images of the subjects had rTmax values ranging from +1.2° C. to +2.6° C. Subject 2 had an rTmax of +2.6° C. at the second imaging time, which may indicate a post infection healing inflammatory state since we postulate that the temperature range for inflammation is +1° C. to <+3° C. In this case series, subjects with maximum wound bed temperatures of ≧+3° C. (FIG. 4) had clinical infection, which is in agreement with prior studies. Additionally the heterogeneity decreased as the infection subsided with the HI index score decreasing substantially. When looking at wound measurements, we found that in the three cases with open wounds, treating the infection facilitated a healing trajectory, in that all of the wounds experienced a size reduction as reported in the literature. The range was wide, from an increase of 2% to a reduction of 73.8% based on wound trace perimeter length, and reductions from 10.8% to 89.9% based on wound trace area. However, Subject 3, with a 10.8% improvement based on wound trace area, was imaged only two days after antibiotics were started. Finally, the heterogeneity and reduction in size similarly represent healing. The HI reduction and wound trace size are correlated.

More research is needed in the form of a prospective study with control groups of normal, inflamed and infected wounds to make additional observations of how thermography can be utilized to identify barriers to healing and ultimately lead to early identification, treatment, and prevention. Additionally, the HI needs to be evaluated on a larger group and compared with perimeter length as a measure of wound improvement and to determine if there are specific HI ranges associated with infection, inflammation, as well as normal healing wounds.

The present invention is equally applicable to assessments within high and low temperature ranges. Different multipliers may be used in either context; however, the principles and teachings of the present invention are equally applicable in any temperature range.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of comparing the heterogeneity of a wound a two discrete times, the method comprising the steps of:
providing an apparatus adapted to capture thermal images of biological material using long wave infrared thermography;
using said apparatus to capture a thermal image of a wound at a first discrete time;
using said apparatus to capture a thermal image of said wound at a second discrete time;
using a computer program to determine the weighted thermal Heterogeneity Index of said wound at said first time;
using a computer program to determine the weighted thermal Heterogeneity Index of said wound at said second time; and
using a computer program to compare the weighted thermal Heterogeneity Index of said wound at said first time to the weighted thermal Heterogeneity Index of said wound at said second time.

2. A method of comparing the heterogeneity of a wound the heterogeneity of a wound and peripheral wound corresponding thereto, the method comprising the steps of:
providing an apparatus adapted to capture thermal images of biological material using long wave infrared thermography;
using said apparatus to capture a thermal image of a wound;
using said apparatus to capture a thermal image of a peripheral wound surrounding said wound;
providing a computer program adapted to determine the weighted thermal Heterogeneity Index of said wound;
using said computer program to determine the weighted thermal Heterogeneity Index of said peripheral wound; and
using a computer program to compare the weighted thermal Heterogeneity Index of said wound to the weighted thermal Heterogeneity Index of said peripheral wound.
